(19) United States
(12) Patent Application Publication
(10) Pub. No.: US 2015/0030578 A1
(43) Pub. Date: Jan. 29, 2015

(54) METHOD FOR PRODUCING ACTIVATED AUTOLOGOUS PLATELET RICH AND PLATELET POOR PLASMA AND METHODS OF USE

(71) Applicants: Bill J. Releford, JR., Inglewood, CA (US); James Hays, Inglewood, CA (US); John Simmonds, Santa Monica, CA (US)

(72) Inventors: Bill J. Releford, JR., Inglewood, CA (US); James Hays, Inglewood, CA (US); John Simmonds, Santa Monica, CA (US)

(21) Appl. No.: 14/338,984

(22) Filed: Jul. 23, 2014

Related U.S. Application Data
(60) Provisional application No. 61/857,669, filed on Jul. 23, 2013.

(51) Int. Cl. A61K 35/14; C12N 5/078

(52) U.S. Cl. CPC A61K 35/19; C12N 5/0644; C12N 2501/00
USPC 424/93.72; 435/2

(57) ABSTRACT

A method and kit to produce activated autologous platelet rich and platelet poor plasma (AAPRPP) and methods of use to treat pain. The method to produce AAPRPP generally comprising (1) obtaining whole blood from patient; (2) centrifuging whole blood in a collection tube; (3) extracting platelet rich and platelet poor plasma mixture from collection tube; and (4) transferring platelet rich and platelet poor plasma mixture to an activation tube containing at least one platelet aggregator and at least one platelet activator to obtain a resulting platelet concentration. The method to treat pain generally comprising producing AAPRPP and extracting contents of activation tube into a standard syringe then injecting AAPRPP at or near the nerve group responsible for the affected anatomical area.

### RELATED APPLICATIONS

This application claims priority to U.S. Provisional Application No. 61/857,669 filed Jul. 23, 2013. The entire contents of the above application are hereby incorporated by reference as though fully set forth herein.

### BACKGROUND

Patients diagnosed with human immunodeficiency virus (HIV) are known to experience disorders of the peripheral nervous system that affects multiple sensory and motor nerves known as neuropathy. Those that experience HIV associated neuropathy may experience symptoms such as painful numbness, tingling, burning and other associated paresthesias in different parts of the body, especially in the upper and lower extremities. HIV associated peripheral neuropathy is the most common neurologic complication related to HIV infections.

Currently, treatments for HIV associated neuropathy include the use of anti-seizure medicines, antidepressants, or analgesics such as opiate drugs. For example, US Patent Application 2009/0281194 to Johns et. al. discloses combinations of cannabinoid receptors and opioids for the purposes of treating HIV associated pain or neuropathy. Further, US Patent Application 2011/0182972 to Bley discloses the use of a pain-relieving patch containing capsaicin or other capsaicin analogs. Many times these treatments are ineffective and costly. As such, there exists a need for a method of treatment that relieves the symptoms of neuropathy and particularly HIV associated peripheral neuropathy. The present invention addresses this need by providing a novel composition of activated autologous platelet rich and platelet poor plasma (AAPRPP), novel process and a kit for preparing AAPRPP and a method of using the AAPRPP to treat neuropathy and particularly HIV associated peripheral neuropathy.

It has been well documented that activated platelets from platelet rich plasma (PRP) as well as platelet poor plasma (PPP) contains numerous growth factors, including those that facilitate peripheral nerve regeneration, neurogenesis and nerve repair. Generally, platelets are activated within the body due to molecules that facilitate activation such as collagen, thromboxane A2, ADP and thrombin. However, there are known methods to activate platelets in PRP outside of the body. One aspect of the present invention is to improve upon these methods of activation to produce higher concentrations of activated platelets containing growth factors from the PRP and PPP for use as a therapeutic agent.

Processes for producing activated PRP and PPP are well known in the prior art, such as those disclosed in US Patent Application 2009/0317439 to Turzi et. al., U.S. Pat. No. 6,398,972 to Blasetti et. al., WIPO Patent Application 2004024198 to Sacchi and US Patent Application 2008/0286379 to Reinecke et. al. However, certain embodiments of the present invention represent a novel process and kit for producing a composition of AAPRPP with an improved concentration of activated platelets containing growth factors that is used in a method of treatment for neuropathy and in particular HIV associated peripheral neuropathy.

In one embodiment of the present invention, a method of treatment delivers a composition of AAPRPP with a high concentration of activated platelets containing growth factors directly to the area affected by neuropathy, resulting in reduction of neuropathy related clinical manifestations such as painful numbness, tingling, burning and other associated paresthesias in different parts of the body, especially in the upper and lower extremities. The method of treatment is easy to use and has the advantages of point of care administration as well as a relatively quick and painless application, which helps facilitate patient compliance with the treatment.

Clinical experiences at the time of filing of this patent application have been overwhelmingly positive, with a majority of the patient population experiencing immediate response to the treatment using AAPRPP by experiencing relief from the clinical manifestations of neuropathy, as shown in “Case Study: Novel Approach to HIV-Associated Neuropathy Platelet Rich Plasma Successful in Treating HIV-Associated Peripheral Neuropathy” Journal of Therapy and Management in HIV Infection, 2013, Volume 1, No. 2, pp. 1-5, which is incorporated by reference herein. Another inherent advantage to the present invention when compared to the use of other therapeutic treatments used to treat peripheral neuropathy, especially HIV associated neuropathy, is that the AAPRPP is derived from the patient's own blood, which eliminates the risk of allergic or adverse reactions to the treatment other than minimal site injection tenderness expected from any injection type treatment.

Additionally, the use of AAPRPP has been shown to be clinically useful in methods to treat pain associated neurological conditions such as multiple schlerosis, cerebral palsy and Charot-Marie Tooth diseases as well as in wound care, dermatology and sports medicine/orthopedics.

### BRIEF SUMMARY OF THE INVENTION

The present invention relates to a novel composition of AAPRPP, a process to produce AAPRPP, a kit to perform the process to produce AAPRPP and use of AAPRPP to treat neuropathy. In the first aspect of the present invention, a novel composition of AAPRPP is disclosed. In the second aspect of the present invention, a novel process for obtaining AAPRPP from whole blood is disclosed. In a third aspect of the present invention, a novel kit for obtaining AAPRPP from whole blood is disclosed. In a fourth aspect of the present invention, a method of using AAPRPP to treat neuropathy and in particular HIV associate peripheral neuropathy, as well as a method to treat pain associated with various inflammatory conditions is disclosed.

### DETAILED DESCRIPTION

The following definitions are intended to apply uniformly throughout this application unless an otherwise expressly set out definition provides a broader definition:

The term “autologous” is intended to describe a method of the invention using a single donor's whole blood and wherein the AAPRPP produced from this donor is intended to be used on the same donor. Using autologous product avoids any issues created by using biological materials from third parties, such as the need to screen to assure the donor is biologically or immunologically compatible with the patient and avoids potential contamination with diseases transmitted by blood such as hepatitis.

The term “thrombin activator” is used to describe any agent that is able to activate thrombin and to trigger coagulation. Examples of thrombin activators include certain cofactors such as sodium or calcium. In practicing the present invention, thrombin activation is partially attributed to the presence of calcium ions. Suitable calcium salts that can be used include, without limitation, CaCO₃ and CaSO₄ with the preferred calcium salt used in this invention as calcium chloride (CaCl₂) available as an injection, USP 10% (Regen Lab, Switzerland).

The term “phlebotomy” is used to describe any technique that allows for the puncture of a vein with a needle for the purpose of drawing blood.

The term “point-of-care” is used to describe clinical services provided to patients in a clinical setting or at bedside.

In one embodiment, the present invention provides an isolated AAPRPP concentrate composition comprising platelets at a concentration of at least 200,000 microliter (mcl), preferably of at least 400,000 mcl of platelets; by using (1) calcium chloride at a concentration of at least 30 mg/mL, preferably of at least 100 mg/mL calcium chloride; and (2) zinc sulfate at a concentration of at least 30 mg/ml, preferably of at least 100 mg/mL zinc sulfate. The AAPRPP may further comprise fibrinogen at a concentration of at least 100 mg/dL, preferably of at least 400 mg/dL. In the preferred embodiment, the concentration of the calcium chloride solution is 100 mg/mL and the amount of calcium chloride solution used is from about 0.5 to about 1.0 mL. In the preferred embodiment, the concentration of the zinc sulfate solution is 100 mg/mL and the amount of zinc sulfate solution used is about 0.003 mL.

In a second embodiment, the present invention provides a kit adapted for producing AAPRPP according to the invention wherein the kit comprises (1) accessories for the phlebotomy, (2) a blood collection tube containing acid-citrate dextrose (ACD) solution and (3) a tube with a solution comprising at least one platelet activator and at least one platelet aggregator to produce AAPRPP. The preferred embodiment of the present invention is to use a platelet activator comprising calcium chloride at a concentration of at least 30 mg/mL, preferably of at least 100 mg/mL calcium chloride. The preferred embodiment of the present invention is to use a platelet aggregator comprising zinc sulfate at a concentration of at least 30 mg/ml, preferably of at least 100 mg/mL zinc sulfate. In the preferred embodiment of the mixture, the concentration of the calcium chloride solution is 100 mg/mL and the amount of calcium chloride solution used is from about 0.5 to about 1.0 mL. In the preferred embodiment of the mixture, the concentration of the zinc sulfate solution is 100 mg/mL and the amount of zinc sulfate solution used is about 0.003 mL.

In the preferred embodiment, the blood collection tube contains solutions typically available in the industry, such as a solution of tri-sodium citrate (22. g/L), citric acid (8.0 g/L) and dextrose (24.5 g/L). Another common solution is tri-sodium citrate (13.2 g/L), citric acid (4.8 g/L) and dextrose (14.7 g/L). (Fisher Scientific, USA)

A third embodiment of the present invention is a process to produce AAPRPP wherein the process comprises the steps of (1) obtaining whole blood from patient; (2) centrifuging whole blood; (3) extracting platelet rich and platelet poor plasma mixture from collection tube; and (4) transferring platelet rich and platelet poor plasma mixture to an activation tube with a mixture comprising a at least one platelet aggregator and at least one platelet activator to produce AAPRPP.

The whole blood is obtained using standard phlebotomy and is collected in a standard vacutainer tube such as those used for blood banking studies, HLA phenotyping and DNA paternity testing.

Several techniques can be used to centrifuge the whole blood to obtain the platelet rich and platelet poor plasma. In the preferred embodiment of the present invention, the whole blood should be centrifuged in a single spin cycle at a speed of at least 3200 rpm, preferably at a speed of at least 4000 rpm and for a time period of at least 4 minutes, preferably for a time period of at least 10 minutes. Although platelet composition varies from patient to patient, applicants have demonstrated centrifugation at 4000 rpm for at least 10 minutes generally results in around a two-fold increase in platelet concentration in the resulting platelet rich and platelet poor plasma mixture.

Once the platelet rich and platelet poor mixture has been separated from the whole blood, it is extracted from the tube used to centrifuge the whole blood and transferred into an activation tube. The activation tube contains a mixture of at least one platelet activator and at least one platelet aggregator to produce AAPRPP. The preferred embodiment of the present invention is to use a platelet activator comprising calcium chloride at a concentration of at least 30 mg/mL, preferably of at least 100 mg/mL calcium chloride. The preferred embodiment of the present invention is to use a platelet aggregator comprising zinc sulfate at a concentration of at least 30 mg/ml, preferably of at least 100 mg/mL zinc sulfate. In the preferred embodiment of the mixture, the concentration of the calcium chloride solution is 100 mg/mL and the amount of calcium chloride solution used is from about 0.5 to about 1.0 mL. In the preferred embodiment of the composition, the concentration of the zinc sulfate solution is 100 mg/mL and the amount of zinc sulfate solution used is about 0.003 mL.

Although calcium chloride is known as a platelet activator, one novel aspect of the present invention is using zinc as a platelet aggregator. Zinc is known to enhance platelet aggregation. In the present invention, the zinc significantly enhances platelet aggregation and presumably results in an increased delivery of activated platelets containing growth factors to the affected area resulting in nerve regeneration, which relieves neuropathy symptoms.

A fourth embodiment of the present invention is a method to treat inflammatory response in the body, the method comprising (1) obtaining whole blood from patient; (2) centrifuging whole blood; (3) extracting platelet rich and platelet poor plasma mixture from collection tube; (4) transferring platelet rich and platelet poor plasma mixture to an activation tube with a mixture comprising at least one platelet aggregator and at least one platelet activator to produce AAPRPP; (5) extracting contents of activation tube into a standard syringe; and (6) injecting AAPRPP at or near the nerve group responsible for the affected anatomical area.

The whole blood is obtained using standard phlebotomy and is collected in a standard vacutainer tube such as those used for blood banking studies, HLA phenotyping and DNA paternity testing.

Several techniques can be used to centrifuge the whole blood to obtain the platelet rich and platelet poor plasma. In the preferred embodiment of the present invention, the whole blood should be centrifuged in a single spin cycle at a speed of at least 3200 rpm, preferably at a speed of at least 4000 rpm and for a time period of at least 6 minutes, preferably for a time period of at least 10 minutes. Although platelet composition varies from patient to patient, applicants have demonstrated centrifugation at 4000 rpm for at least 10 minutes generally results in around a two-fold increase in platelet concentration in the resulting platelet rich and platelet poor plasma mixture.

Once the platelet rich and platelet poor mixture has been separated from the whole blood, it is extracted from the tube used to centrifuge the whole blood and transferred into an activation tube. The activation tube contains a mixture of at least one platelet activator and at least one platelet aggregator to produce AAPRPP. The preferred embodiment of the present invention is to use a platelet activator comprising calcium chloride at a concentration of at least 30 mg/mL, preferably of at least 100 mg/mL calcium chloride. The preferred embodiment of the present invention is to use a platelet aggregator comprising zinc sulfate at a concentration of at least 30 mg/ml, preferably of at least 100 mg/mL zinc sulfate. In the preferred embodiment of the mixture, the concentration of the calcium chloride solution is 100 mg/mL and the amount of calcium chloride solution used is from about 0.5 to about 1.0 mL. In the preferred embodiment of the composition, the concentration of the zinc sulfate solution is 100 mg/mL and the amount of zinc sulfate solution used is about 0.003 mL.

Although calcium chloride is known as a platelet activator, one novel aspect of the present invention is using zinc as a platelet aggregator as zinc is known to enhance platelet aggregation. In the present invention, the zinc significantly enhances platelet aggregation and presumably results in an increased delivery of activated platelets containing growth factors to the affected area resulting in nerve regeneration, which relieves neuropathy symptoms.

The AAPRPP composition is administered at the point of care at preferably no more than one hour after preparation of the AAPRPP with the composition preferably administered immediately after preparation of the AAPRPP. Preferably the injection is subcutaneous or intramuscular. The injection site is preferably at or near the distribution of nerve groups responsible for the clinical manifestations of the neuropathy experienced by the patient.

Typically, the AAPRPP can be prepared using the kit of the present invention and the process of the present invention in about fifteen (15) minutes, making the treatment possible at point of care and without significant wait time after drawing the whole blood.

There are several protocols used to produce AAPRPP that are customized based on the therapeutic area.

Protocol: I (Used to Treat Nerve and Vertebral Disc Inflammation/Injuries)
20 cc of blood obtained
Placed in centrifuge with a counterbalance tube
Device set for 10 min at 4000 rpm
Plasma activated with 1 ml CaCl+ZnSO4
Yield 10 cc of PRP+PPP or 4 cc PRP

The spin protocol 1: allows better separation of the plasma cells i.e red, and white blood cells. Plasma products base are modified based on cell type concentration. These modifications are listed below in detail.
PlasmaNeu: 1: Low molecular-weight, low viscosity, low leukocytes concentration, high PRP (Acute nerve/disc injury)
PlasmaNeu: 2: Low molecular-weight, low viscosity, moderate leukocytes concentration, high PRP (Sub-acute nerve/disc injury)
PlasmaNeu: 3: Low molecular-weight, low viscosity, high leukocytes concentration, high PRP concentration (Chronic nerve/disc injury)

Protocol: II (Used to Treat Ligament and Tendon Inflammation/Injury)
20 cc of blood obtained
Placed in centrifuge with a counterbalance tube
Device set for 10 min at 3300 rpm
Plasma activated with 0.5 ml CaCl+ZnSO4
Yield 10 cc of PRP+PPP or 4 cc PRP

The spin protocol 2: allows better separation of the plasma cells i.e red, and white blood cells. Plasma products are modified based on cell type concentration. These modifications are listed below in detail.
PlasmaPro: 4: Low molecular-weight, low viscosity, low leukocytes concentration, PRP@PPR (Acute tendon/ligament injury)
PlasmaPro: 5: Low molecular-weight, low viscosity, moderate leukocytes concentration, high PRP concentration (Sub-acute tendon/ligament injury)
PlasmaPro: 6: Low molecular-weight, low viscosity, high leukocytes concentration, high PRP concentration (Chronic tendon/ligament injury)

Protocol: III (Used to Treat Muscle Inflammation/Injury)
20 cc of blood obtained
Placed in centrifuge with a counterbalance tube
Device set for 8 min at 3300 rpm
Plasma activated with 0.5 ml CaCl+ZnSO4
Yield 10 cc of PRP+PPP or 4 cc PRP

The spin protocol 3: allows better separation of the plasma cells i.e red, and white blood cells. Plasma products are modified base on cell type concentration and viscosity. These modifications are listed below in detail.
PlasmaNeu: 4: High molecular-weight, high viscosity, low leukocytes concentration, high PRP (Acute muscle injury)
PlasmaNeu: 5: High molecular-weight, high viscosity, moderate leukocytes concentration, high PRP (Sub-acute muscle injury)
PlasmaNeu: 6: High molecular-weight, high viscosity, high leukocytes concentration, high PRP (Chronic muscle injury)

Protocol: IV (Used to Treat Cartilage Inflammation/Injury)
20 cc of blood obtained
Placed in centrifuge with a counterbalance tube
Device set for 6 min at 3200 rpm
2^{nd} spin set for 4 min at 3300 rpm
Plasma activated with 0.5 ml CaCl+ZnSO4
Yield 10 cc of PRP+PPP or 5 cc PRP

The spin protocol 4 allows better separation of the plasma cells i.e red, and white blood cells. Plasma products are modified based on cell type concentration and viscosity. These modifications are listed below in detail.
PlasmaPro: 1: High molecular-weight, high viscosity, low leukocytes concentration, PRP and PPR (Acute cartilage injury)
PlasmaPro: 2: High molecular-weight, high viscosity, moderate leukocytes concentration, high PRP concentration (Sub-acute cartilage injury)
PlasmaPro: 3: High molecular-weight, high viscosity, high leukocytes concentration, high PRP concentration (Chronic cartilage injury)

Clinical efficacy has been shown in various therapeutic areas related to inflammatory responses. Pain management using AAPRPP have been shown in several case studies set forth below.

Case Study

The purpose of this case study is to explain and showcase a novel approach to treat diabetic wounds using harvested dermal cells combined with autologous fibrin matrix.

The purpose of this case study is to explain and showcase a novel approach to treat and cure proximal hamstring syndrome with AAPRPP blood therapy.

The most common hamstring injuries are acute or chronic muscle strains, they are also vulnerable to tendonitis at their origin, an injury termed high hamstring tendinopathy or proximal hamstring tendonitis. While rare, this injury is difficult to treat and can become a prolonged and chronic problem. The relatively limited scientific and medical reports extant are fairly recent, and as such, there are no solid numbers on what percentage of runners come down with it.

The hamstrings run from the top of your tibia, just behind your knee, up along the back side of your thigh and towards your pelvis. While one branch of the hamstrings attaches to the femur, the rest course up your thigh and underneath your gluteal muscles, attaching to the pelvis at a bony prominence called the ischial tuberosity. These twin “peaks” of bone are sometimes referred to as your “sitting bones,” as they support much of your weight while sitting, especially on hard surfaces. The junction between the tendons of the hamstrings and the ischial tuberosity is the area affected by high hamstring tendinopathy.

Sufferers will complain of pain local to the ischial tuberosity when running, especially when accelerating and sustained faster paced running The pain will most likely be an intense ache in nature, rather than sharp or stabbing. Due to the anatomical proximity to the common hamstring origin, the sciatic nerve can sometimes be affected, which can cause referred pain into the posterior thigh. Once aggravated, sitting on solid surfaces can also be uncomfortable, as can direct palpation and pressing onto the ischial tuberosity manually.

A 38 year old female was referred to the Plasmagenix Medical Corporation for an evaluation and treatment for chronic proximal hamstring syndrome. Prior to referral, the patient was diagnosis with chronic proximal hamstring syndrome via objective evidence on MRI and her clinical presentation. The patient's subjective and objective findings demonstrated a Nirschl Phase Rating of 4. The patient was treated with 4-5 months of intense physical therapy and one cortisone injection with no improvement. The patient was evaluated by a Certified Plasmagenix Medical Provider and was indicated for treatment with AAPRPP blood therapy. After informed consent a Right arm tourniquet was applied and the targeted vein identified. The area was then prepped in the usual aseptic manner. Using a butterfly blood collection set with a 21 gauge needle, approximately 18 mL of whole blood was collected from the patient. Approximately 12 mL of whole blood was deposited into the centrifuge container under sterile technique. The centrifuge was counterbalanced with 18 mL of normal saline in a similar container.

The centrifuge was then set at 10 minutes at 3600 RPM. After 10 minutes had elapsed, a thin buffy coat layer representing platelet rich plasma could be identified in the centrifuge tube. Using the plasma extractor, all plasma was removed down to the layer of platelet rich plasma. Using a 12 mL syringe approximately 12 mL of platelet rich plasma was extracted from the centrifuge tube.

The platelet rich plasma was then taken into the examination room. The patient was placed in the prone position on the treatment table. The skin over and surrounding the treatment area was cleaned with Betadine. The area was covered with sterile drapes, leaving a small window opening for needle placement. Ultrasound was used to identify the boney landmarks of the right ischial tuberosity and the planned needle approach. The skin, subcutaneous tissue, and muscle within the planned needle approach were anesthetized with 1% Lidocaine. All injected medications were preservative free. With ultrasound guidance an 18 gauge needle introduced to the Right biceps femoris, semimembranosus, and semitendinosus tendons. Percutaneous needle tenotomy was performed by way of multiple fenestrations to the Right biceps femoris, semimembranosus, and semitendinosus tendons. Finally, the treatment solution, consisting of 6 cc of platelet rich plasma and platelet poor plasma activated with Plasmagenix's proprietary compound was injected in the Right biceps femoris, semimembranosus, and semitendinosus tendons. All injected medications were preservative free. Sterile technique was used throughout the procedure.

This patient underwent two therapeutic procedures two weeks apart with concurrent physical therapy treatment. The patient was re-evaluated two months after the commencement of her treatment, and presented pain-free with all physical activities.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the method (and components of the individual operating components of the method) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections might be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

What is claimed is:

1. A method to produce activated autologous platelet rich and platelet poor plasma (AAPRPP) comprising:
(1) obtaining whole blood from patient;
(2) centrifuging whole blood in a collection tube;
(3) extracting platelet rich and platelet poor plasma mixture from collection tube; and
(4) transferring platelet rich and platelet poor plasma mixture to an activation tube containing at least one platelet aggregator and at least one platelet activator to obtain a resulting platelet concentration.

2. The method according to claim 1 wherein the platelet aggregator is calcium chloride and the platelet activator is zinc sulfate.

3. The method according to claim 2 wherein the calcium chloride is in a solution at a concentration of at least 30 mg/mL and zinc sulfate is in a solution at a concentration at least 30 mg/mL.

4. The method according to claim 2 wherein the calcium chloride is in a solution at a concentration of about 100 mg/mL.

5. The method according to claim 2 wherein the zinc sulfate is in a solution at a concentration of about 100 mg/mL.

6. The method according to claim 4 wherein the amount of solution of calcium chloride is about 0.5 to about 1.0 mL.

7. The method according to claim 5 wherein the amount of solution of zinc sulfate is about 0.003 mL.

8. The method according to claim 1 wherein the resulting platelet concentration is from about 200,000 to about 400,000 platelets per microliter (mcl)

9. A method of using activated autologous platelet rich and platelet poor plasma (AAPRPP) to treat pain comprising:
(1) obtaining whole blood from a patient;
(2) centrifuging whole blood in collection tube;
(3) extracting platelet rich and platelet poor plasma mixture from collection tube;
(4) transferring platelet rich and platelet poor plasma mixture to an activation tube containing at least one platelet aggregator and at least one platelet activator to produce AAPRPP;
(5) extracting contents of activation tube into a standard syringe; and
(6) injecting AAPRPP at or near the nerve group responsible for the affected anatomical area.

10. The method of claim 9 wherein the cause of the pain is selected from the group comprising HIV-related neuropathy, diabetic neuropathy, neuropathy, nerve injury, vertebral disc injury, ligament injury, tendon injury, muscle injury, cartilage injury, skin wounds, neurological disorders or combinations thereof.

11. The method of claim 9 further comprising adding fibrinogen in a solution at a concentration from about 100 mg/dL to about 400 mg/dL to the AAPRPP prior to injection.

12. The method according to claim 9 wherein the platelet aggregator is calcium chloride and the platelet activator is zinc sulfate.

13. The method according to claim 9 wherein after activation the platelets are in a solution at a concentration from about 200,000 to about 400,000 platelets per microliter (mcl).

14. A kit adapted for producing activated autologous platelet rich and platelet poor plasma (AAPRPP) wherein the kit comprises:
(1) accessories for phlebotomy;
(2) a blood collection tube containing acid-citrate dextrose (ACD) solution; and
(3) a tube containing a composition to activate autologous platelet rich and platelet poor plasma comprising at least one platelet aggregator and at least one platelet activator to produce AAPRPP.

15. The kit according to claim 14 wherein the platelet aggregator is calcium chloride and wherein the platelet activator is zinc sulfate.

16. The kit according to claim 15 wherein the calcium chloride is in a solution at a concentration of at least 30 mg/mL and zinc sulfate is in a solution at a concentration at least 30 mg/mL.

17. The kit according to claim 15 wherein the calcium chloride is in a solution at a concentration of about 100 mg/mL.

18. The kit according to claim 15 wherein the zinc sulfate is in a solution at a concentration of about 100 mg/mL.

19. The kit according to claim 18 wherein the amount of solution of calcium chloride is about 0.5 to about 1.0 mL.

20. The kit according to claim 19 wherein the amount of solution of zinc sulfate is about 0.003 mL.
